(19) United States
(12) Patent Application Publication
(10) Pub. No.: US 2023/0136390 A1
(43) Pub. Date: May 4, 2023

(54) HIGHLY EFFECTIVE, SILICA-FREE, STORAGE STABLE DENTAL ETCHING GEL

(71) Applicant: VOCO GmbH, Cuxhaven (DE)

(72) Inventors: Hauke Kelch, Cuxhaven (DE); Wigand Krumme, Cuxhaven (DE); Manfred Thomas Plaumann, Cuxhaven (DE)

(21) Appl. No.: 18/052,376

(22) Filed: Nov. 3, 2022

(30) Foreign Application Priority Data
Nov. 4, 2021 (DE) 10 2021 128 685.9

(51) Int. Cl. A61K 6/69; A61K 6/65; A61C 5/00

(52) U.S. Cl. CPC A61K 6/69; A61K 6/65; A61C 5/00

(57) ABSTRACT

The present invention relates to a dental etching composition comprising phosphoric acid, water and urethane-urea compound(s), to the use of said dental etching composition for etching the hard substance of the tooth, to a dental etching composition for use in a therapeutic method of etching the hard substance of the tooth in the course of filling treatment, and to a kit comprising a dental etching composition.

### CROSS-REFERENCE TO RELATED APPLICATIONS

This application claims priority to German Patent Application No. 10 2021 128 685.9, filed Nov. 4, 2021, which is herein incorporated by reference in its entirety.

### FIELD OF THE DISCLOSURE

The present invention relates to a dental etching composition, to the use of said dental etching composition for etching the hard substance of the tooth, to a dental etching composition for use in a therapeutic method of etching the hard substance of the tooth in the course of filling treatment, and to a kit comprising a dental etching composition.

### BACKGROUND OF THE DISCLOSURE

### Dentine and Enamel as Hard Substance of the Tooth

The hard substance of the tooth consists of enamel and dentine. While the enamel consists to an extent of 95% by weight of inorganic substance, to an extent of 4% by weight of water and to an extent of 1% by weight of organic matrix constituents, the dentine is much less significantly mineralized. It forms the main mass of the tooth, constitutes a vital hard tissue, and imparts the specific shape to the tooth. The dentine encloses the dental pulp and is coated coronally by enamel and in the root region by cement. The dentine forms from the dental papilla and is comparable to bone in terms of its chemical composition. It is fundamentally different from the enamel. Dentine consists to an extent of 70% by weight of inorganic constituents, in particular of hydroxyapatite, to an extent of about 20% by weight of organic constituents and to an extent of about 10% by weight of water. Owing to its high proportion of organic substances, dentine is highly elastic and formable.

The mineralized portion contains essentially calcium and phosphorus, variable concentrations of fluoride, small amounts of carbonates and magnesium, and some trace elements. The organic matrix consists to an extent of more than 90% of type I collagen. The remainder of organic substances is composed of non-collagen base structure, examples being proteins, lipids, citrates and lactates.

In terms of its morphological structure, dentine is composed of the dentinal canals including the periodontoblastic space, the odontoblasts with their prolongations, the peritubular dentine, the intertubular dentine and the mantle dentine. Intertubular dentine is the network consisting of type I collagen, incorporating the platelet-shaped hydroxyapatite crystals and dentine liquor. The tubuli contain the peritubular dentine, a collagen fiber tube, odontoblast prolongations and dentine liquor. Peritubular dentine, which lines the canal walls, is homogeneous and dense and the most highly mineralized of all the dentine structures.

The dentinal canals decrease in number and diameter from the dental pulp to the enamel-dentine boundary. From an average of 45 000/mm² at the dental pulp-dentine boundary, there is already a reduction in this number to 20 000/mm² at a distance of 3 mm from the dental pulp. The diameter is reduced from 2 to 3 μm at the dental pulp to 0.5 to 0.9 μm at the enamel-dentine boundary.

The odontoblasts, i.e., the dentine-producing cells of the tooth, lie at the inner surface of the dentine. After differentiation, they are no longer capable of dividing, but are capable of lifelong formation of secondary and tertiary dentine. The odontoblast prolongations run in the dentinal canals. Each prolongation is surrounded by tissue fluid, the dentine liquor, that fills the periodontoblastic space. The prolongations permeate the entire dentine and may have a length of up to 5000 μm. Side branches that reach into the intertubular dentine are in contact with the lateral branches of the neighboring prolongations. The periodontoblastic space between the odontoblasts consists for the most part of tissue fluid. The intertubular dentine separates the individual dentinal canals. It is less mineralized than the peritubular dentine.

In the preparation of a tooth, the dentinal channels are inevitably opened. The result is an open dentine wound which, on account of the internal pulp pressure, allows dentine liquor to flow out along the dentinal canals. This phenomenon is also referred to as intrinsic moisture. For this reason, dentine cannot be dried absolutely in vivo.

If dentine is being prepared (for example with rotating instruments), the result is a 1.5 μm-thick smear layer consisting of particles, having a size of 0.5 to 1.5 μm, of hard tooth substance, constituents of collagen, blood and saliva, and bacteria and the metabolism products thereof. This layer results firstly in plugging of the dentinal channels; secondly, the smear layer covers the area of the prepared dentine, which lowers the permeability of the dentine. This smear layer cannot be rinsed away or removed mechanically. It thus makes it difficult to adapt the restoration materials on the tooth surface and impairs the adhesion of plastics. However, the smear layer can be removed by chemical pretreatment of the dentine.

By contrast, the inorganic substance of the enamel consists mainly of calcium phosphate in the form of hydroxyapatite [Ca₁₀(PO₄)₆(OH)₂], but this cannot be regarded as a stoichiometric pure material by virtue of inclusions of carbonate, fluoride, sodium, magnesium, potassium and other ions. Internal substitution reactions can result in formation of fluorapatite or fluoridated hydroxyapatite. The crystal structures of these compounds are more acid-stable than those of pure hydroxyapatite.

The proportion of inorganic compounds varies, according to the method of analysis and sampling site, between 93% and 98% by weight. Water as the second greatest constituent varies between 1.5% and 4% by weight. On account of the different concentrations of the enamel composition at the various sites in the tooth, there is a decrease in the amount of fluoride, iron, tin, chlorine and calcium from the surface with increasing depth, with an increase in the fluoride concentration again at the enamel-dentine boundary. By contrast, the concentrations of water, carbonate, magnesium and sodium increase from the enamel surface to the enamel-dentine boundary.

The water is present both in crystalline form, bound as hydration shell on the apatite crystals, and in loose form, fixed on the organic enamel matrix. The loosely bound water can evaporate when heated and be absorbed again with supply of moisture. In the case of this flow of liquid, the enamel acts as a molecular sieve, with ions being able to migrate both out of the enamel and into it.

The apatite crystals have a hexagonal cross section and have an average length of 169 nm, an average width of 40 to 70 nm and an average thickness of 26 nm. Even though, in chemical terms, they are likewise calcium phosphates of the apatite type, they are very much larger than crystallites of the same type but of different biological origin. About 100 enamel crystallites are associated in cross section, and form the enamel prisms or rods that extend from the enamel-dentine boundary to the surface. The crystallites in the core of the prisms are aligned with their longitudinal axis parallel to the longitudinal axis of the respective prism.

The totality of the crystallites is embedded into an organic matrix in gel form. The organic substances in the enamel are predominantly proteins, lipids, and traces of carbohydrates and organic acids. All crystallites are additionally surrounded by a hydration shell.

The prisms are in turn embedded in an interprismatic substance which is also formed from enamel crystallites. There are no differences with regard to inorganic content between prisms and interprismatic zones; both consist of densely adjoining crystallites. The microscopically verifiable structuring in prismatic and nonprismatic components is merely a consequence of the crystal arrangement. The crystallites of the interprismatic substance form almost a right angle with the longitudinal axis of the prisms.

The enamel is only of limited permeability to ions, water, dyes and alcohol. It has a high modulus of elasticity and low tensile strength.

Both enamel and dentine are thus highly complex structures that are also viewed differently with regard to dental adhesive treatment.

### Enamel Adhesion

The adhesion of plastics to the enamel is based predominantly on micromechanical retention, and to a lesser degree on chemical adhesion. The principle of predominantly mechanical enamel adhesion was described for the first time in 1955 and is nowadays a standard method in adhesive restorations in dental practice under the name “acid etching methodology” or “enamel etching methodology”. On account of the different solubility of the individual enamel prism structures, it is possible to achieve a microretentive etching pattern with 30% to 40% phosphoric acid. A low-viscosity plastic can penetrate as adhesion promoter into this etching pattern of the enamel and hence ensure bonding to the filling composite via good interdigitation. In the case of fissure sealing, the pattern created by the enamel etching is sufficient to achieve an adequate bond between enamel and the sealing material even without an adhesion promoter.

In the case of small fillings in the enamel region, micromechanical adhesion is so good that polymerization shrinkage can be absorbed completely. In order not to overstress the adhesive bond, in the case of larger fillings, attempts are made by layered application and separate curing to compensate for the shrinkage of the material. In general, the bond strength to the enamel is sufficient to prevent the occurrence of marginal gaps resulting from the polymerization.

During the use of the phosphoric acid, there is conversion of the enamel apatite to brushite, and the formation of a nonspecific retentive etching pattern, the structures of which are described as villi, tags, gaps, excrescences or micropores. The enormous increase in surface area results in an increase in surface energy and hence a rise in wettability of the etched enamel.

Optimal acid action is achieved only when the enamel region to be etched has been freed of all plaque and calculus residues. In general, an about 35% phosphoric acid is used as etching liquid, which reduces the uppermost enamel layer by 5 to 10 μm and exposes the prism structure down to a depth of 30 μm. Three different etching pattern types are observed by microscope:

primary demineralization of the central regions of the enamel prisms

demineralization of peripheral prism regions

simultaneous demineralization of the prism centers and the prism periphery

The phosphoric acid should generally be allowed a contact time of about 30 seconds and is then rinsed off thoroughly. The enamel is subsequently dried.

Restoration of the tooth with high-viscosity composite materials thus requires an adhesion promoter in order to assure micromechanical anchoring to the enamel. This adhesion promoter is also referred to as sealer, liner, primer, adhesive or bonding agent.

### Dentine Adhesion

The adhesive bonding of hydrophobic composite materials and dentine is considerably more difficult and more complex by virtue of the tubular microstructure, the intrinsic moisture and the higher content of organic material compared to the enamel. Nevertheless, there have been innumerable developments here, and so it is nowadays possible even to supply dentine-bounded regions with composite.

The mechanism of adhesion of the dentine adhesives is likewise based mainly on micromechanical anchoring to the dentine (called a hybrid layer). The anchoring of the adhesion promoter is achieved by interdigitation and chain formation after organic and inorganic constituents have been leached out of the dentine by means of acidic etching preparations.

Various options are specified for the mechanically retentive anchoring between hydrophobic plastic and the moist dentine surface:
formation of villi by means of polymerized resin in the tubuli with length up to 50 μm
interdigitation in microretentions of demineralized dentine
chain formation with exposed collagen with inclusion of undissolved apatite to form a hybrid layer

The monomer mixtures that have penetrated into the dentine tubuli, after curing, form plastic tags. The bonding of the tags to the demineralized peritubular dentine results in an improvement in bond strength.

The penetration of the conditioned dentine surface with an adhesive results, after curing, in what is called a hybrid layer or “plastic-dentine interdiffusion zone”.

This plastic-permeated dentine layer is thought to make a greater contribution to dentine adhesion than the plastic tags in the dentine tubuli. On account of polymerization shrinkage, the plastic tags do not line the walls of the canals and, as a result of the presence of the dentine liquor, there is incomplete polymerization of the tags. The dentine liquor also prevents deep penetration of the plastic.

Chemical components also seem to play a minor role in the bonding mechanism. The reactive group on the adhesion promoter can interact with the inorganic constituents of the dentine (especially Ca²⁺) and with the organic groups of collagen (amino and hydroxyl groups).

### Total Etch

Dentine etching and hence smear layer removal is referred to as conditioning. Etchants used are EDTA solutions, phosphoric acid (10% to 40%), maleic acid (10%), citric acid (10%) or nitric acid (2.5%). The conditioning agent is to be rinsed off again after a defined contact time. According to the concentration of the acid, there is partial or total dissolution of the smear layer. The additional demineralization of the dentine leads, through selective removal of calcium phosphates from the superficial dentine (1 to 7.5 μm), to exposure of collagen fibers. This network of collagen fibers has lost its mineral support and, after excessive drying of the dentine, collapses onto the underlying dentine like a dense bundle. This is the reason why merely excess water is removed from the dentine surface nowadays, but the dentine is not completely blown dry. This method, known as the “moist bonding technique” or “wet bonding technique”, by virtue of the remaining water, ensures that the intrafibrillar cavities in the collagen fiber network are kept open. It is thus possible for hydrophilic monomers applied later on to penetrate through the demineralized collagen network and—through subsequent polymerization—to bring about micromechanical anchoring. There is thus simultaneously also exposure of the tubuli system and etching of the peritubular dentine.

What is generally used nowadays is a 35% to 40% phosphoric acid solution, which is employed by the “total etch technique”. This involves conducting simultaneous enamel and dentine etching. The acid should have a contact time on the dentine of no longer than 15 to 20 seconds since collagen denaturing and excessive dentine permeability should be avoided. This would lead to reduced promotion of adhesion and later have an adverse effect on the dental pulp.

The procedure nowadays is to commence with the application of the etching gel on the enamel. After a contact time of 15 seconds, the acid is then further applied to the dentine, where it is able to act for a further 15 seconds. The total contact time of 30 seconds on the enamel is necessary to achieve an adequate etching pattern.

At the start of a clinically successful dental adhesive treatment is thus the conditioning of enamel and dentine with an etchant.

The acid was originally diluted with water in order to obtain the desired concentration. However, this results in a fluid solution that cannot be applied accurately. The aqueous acid solutions additionally have the disadvantage that they can run away over the surfaces of the teeth in an uncontrolled manner and—if the dentist is not using a dental dam for desiccation—can easily attack the soft tissue of the mouth and hence severely injure the patient.

In order that the intended area can exclusively be etched accurately, the etchant should have a relatively high viscosity and ideally a thixotropic effect. In order to raise the viscosity of etchants, silica is frequently added, so as to form a gel. The etching gel can be applied accurately and prevent running.

The term “gel” is sharply defined; it is subject to the condition that tan δ<1, where the loss factor tan δ=G″/G′ (loss modulus/storage modulus).

U.S. Pat. No. 4,802,950 to T. P. Croll, entitled “Enamel bonding etchant and procedure”, discloses a pasty etching gel comprising an aqueous solution with 35% to 50% phosphoric acid, fumed silica and abrasive silicon carbide particles. The silica is used here as thickener in order to obtain a gel.

U.S. Pat. No. 6,753,001 B2 and U.S. Pat. No. 6,537,563 B2 to Pentron, entitled “Dental acid etchant composition and method of use”, also use silica for production of an etching gel. These patents protect a composition consisting of an aqueous acid solution and a colloidal nanoscale silica sol in an amount of 3% to 20% by weight based on the overall composition.

However, the silica has the significant disadvantage of low water retention. The effect of this is that water can evaporate and hence increase the acid concentration and the viscosity and/or the consistency of the etchant over time. This will inevitably lead to problems in clinical practice. It is therefore advantageous to use an etching gel that does not contain any silica.

Etching compositions without silica are already known from the prior art.

U.S. Pat. No. 5,954,996 to Centrix, entitled “Dental etch and packaging therefore”, claims the composition of an etchant comprising an acid and anhydrous glycerol in an amount of 10% to 40% by weight based on the composition. Since this composition does not include any excess water, there is no change in acid concentration and viscosity overtime. However, this etchant is not a gel, but rather a liquid. A further question that arises is how this composition is supposed to bring about an etching pattern in the absence of a protic solvent (water) when the dentist desiccates the site in question with a dental dam.

US 2012/0161067 A1 to Far Eastern New Century Corporation, entitled “Dental etching gel composition and method of use thereof”, uses carboxymethylcellulose to increase viscosity. The composition claimed consists of a 37% aqueous phosphoric acid solution and carboxymethylcellulose in amounts of 0.5% to 7% by weight, where the viscosity of the carboxymethylcellulose is about 100 to about 2000 cPs when it is dissolved in an aqueous solution at 1% by weight and has an average level of replacement of sodium salts in the molecular formula of about 21% to 33%. One would expect the acid to attack and degrade the cellulose overtime.

U.S. Pat. No. 6,321,667 B1, entitled “Methods of etching hard tissue in the oral environment”, discloses an etchant containing 17% to 40% by weight of polyoxyalkylene polymer. The compositions show a rise in viscosity at elevated temperature.

U.S. Pat. No. 6,027,341 to Peridoc AB, entitled “Dental cavity conditioning”, claims a composition in which the dentine is etched with EDTA, and the enamel with phosphoric acid and/or citric acid. The method is preferably conducted with thickened compositions. For this purpose, cellulose and derivatives of cellulose, proteins or glycoproteins are used to increase viscosity. As above, it is to be expected that the thickeners will be attacked and degraded by the acids over time.

WO 2007/131725 A1 and EP 2 108 356 A1 discloses hydrochloric acid-containing etching compositions for treatment of enamel lesions.

U.S. Pat. No. 5,722,833 describes the conditioning of dental ceramic surfaces with hydrofluoric acid-containing etching compositions.

WO 2015/142392 A1, entitled “Dental etchant compositions comprising one or more dentin collagen cross-linking agents”, discloses dental etching compositions. These may contain phosphoric acid, maleic acid or citric acid as acid component.

### BRIEF DESCRIPTION OF THE DISCLOSURE

The aim of the present invention was thus that of providing storage-stable, highly active etching gels that have sufficiently high viscosity in order to assure accurate application. On the other hand, the thickeners used are to overcome the disadvantages from the prior art. For instance, they should firstly be acid-stable and secondly have sufficiently high water retention capacity.

In one aspect, the present disclosure is directed to a dental etching composition comprising:
A) an acid selected from the group consisting of phosphoric acid, hydrochloric acid, hydrofluoric acid, maleic acid and citric acid,
B) water and
C) one or more urethane-urea compounds.

In one aspect, the dental etching composition additionally comprises D) one or more water-miscible solvents.

In one aspect, the dental etching composition additionally comprises E) colorants.

In one aspect, the dental etching composition comprises

A) the acid (A) in an amount of 10% to 45% by weight, preferably of 30% to 42% by weight,

B) water (B) in an amount of 30% to 60% by weight, preferably of 40% to 60% by weight,

C) the urethane-urea compounds (C) in an amount of 5% to 20% by weight, preferably of 5% to 15% by weight,

D) the water-miscible solvents (D) in an amount of 0% to 20% by weight, preferably of 1% to 15% by weight, and

E) the colorants (E) in an amount of 0% to 5% by weight, preferably of 0.0001% to 1% by weight,

based in each case on the overall composition.

In one aspect, the dental etching composition is essentially free of fumed silica, preferably essentially free of silica particles, more preferably essentially free of inorganic solids, and/or does not contain any further constituents apart from (A), (B), (C), (D) and (E).

In one aspect, the dental etching composition has a loss factor tan δ of less than 1 and/or a viscosity in the range from 0.1 to 200 Pa*s, preferably from 0.5 to 150 Pa*s, more preferably from 1 to 100 Pa*s, most preferably from 1 to 50 Pa*s, and which preferably even after storage at 23° C. for 6 months has a loss factor tan δ of less than 1 and/or a viscosity in the range from 0.1 to 200 Pa*s, preferably from 0.5 to 150 Pa*s, more preferably from 1 to 100 Pa*s, most preferably from 1 to 50 Pa*s.

In one aspect, the disclosure is directed to a method for etching of the hard substance of a tooth, comprising etching the hard substance of the tooth with a dental etching composition of the present disclosure.

In one aspect, the method comprises the steps of:
i) optionally desiccating the tooth to be treated, preferably with a dental dam,
ii) applying a dental etching composition of the present disclosure to the hard substance of the tooth to be treated,
iii) allowing a contact time of the dental etching composition to achieve an etching effect on the hard substance of the tooth,
iv) rinsing off the dental etching composition,
v) applying a dental primer composition and/or adhesive composition to the etched hard substance of the tooth,
vi) optionally polymerizing the dental primer composition and/or adhesive composition,
vii) applying a dental restoration composition and
viii) polymerizing the dental restoration composition.

In one aspect, the present disclosure is directed to a kit comprising a dental etching composition of the present disclosure, a dental primer composition and/or adhesive composition, and optionally a dental restoration composition.

### DETAILED DESCRIPTION OF THE DISCLOSURE

The present invention relates to a dental etching composition comprising phosphoric acid, water and urethane-urea compound(s), to the use of said dental etching composition for etching the hard substance of the tooth, to a dental etching composition for use in a therapeutic method of etching the hard substance of the tooth in the course of filling treatment, and to a kit comprising a dental etching composition.

It has been found in accordance with the invention that, surprisingly, it is possible to obtain storage-stable, silica-free, highly effective etching gels when these include particular types of urethane-urea compounds as thickeners.

More particularly, the object is achieved by a dental etching composition comprising
A) an acid selected from the group consisting of phosphoric acid, hydrochloric acid, hydrofluoric acid, maleic acid and citric acid,
B) water, and
C) one or more urethane-urea compounds.

The acid (A) is preferably phosphoric acid or hydrochloric acid, more preferably phosphoric acid.

Suitable urethane-urea compounds (C) and the syntheses thereof are described in patent specifications EP 0 006 252 B1, EP 1 048 681 B1, EP 1 188 779 B1, EP 1 396 510 B1, EP 2 370 489 B1, EP 2 475 699 B1 and EP 3 328 909 B1, which are herein incorporated by reference.

In a preferred embodiment, the urethane-urea compounds (C) conform to the formula

R¹—O—C(═O)—NH—R²—NH—C(═O)[—NH—R³—NH—C(═O)—NH—R²—NH—C(═O)]ₓ—OR¹

in which
R¹ is an n-alkyl radical having 4 to 22 carbon atoms, a branched alkyl radical having 4 to 22 carbon atoms, an alkenyl radical having 3 to 18 carbon atoms, a cycloalkyl radical having 3 to 20 carbon atoms, an aryl radical having 6 to 12 carbon atoms, an arylalkyl radical having 7 to 12 carbon atoms, a radical of the formula CₘH₂ₘ₊₁(O—CₙH₂ₙ)ₚ—, CₘH₂ₘ₊₁(OOC—C₂H₂ᵥ)ₚ—, or R⁵—C₆H₄(O—CₙH₂ₙ)ₚ—, wherein m=1 to 22, n=2 to 4, p=1 to 15, v=4 or 5, and R⁵ is an alkyl radical having 1 to 12 carbon atoms, and where different R¹ radicals may be the same or different,
R² is a branched or unbranched alkylene radical having 4 to 22 carbon atoms, alkenylene radical having 3 to 18 carbon atoms, alkynylene radical having 2 to 20 carbon atoms, cycloalkylene radical having 3 to 20 carbon atoms, cycloalkenylene radical having 3 to 20 carbon atoms, arylene radical having 6 to 12 carbon atoms, or arylalkylene radical having 7 to 14 carbon atoms, where different R² radicals may be the same or different,
R³ is

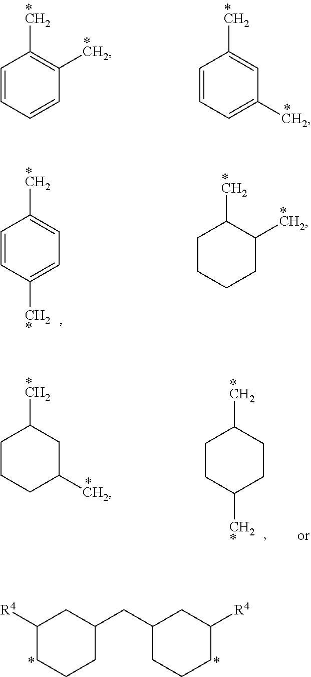

with R⁴=CH₃ or H, where different R³ radicals may be the same or different, and
x is an integer from 1 to 100.

In a particularly preferred embodiment,
R¹ is an n-alkyl radical having 4 to 10 carbon atoms, a branched alkyl radical having 4 to 10 carbon atoms, an alkenyl radical having 3 to 10 carbon atoms, a cycloalkyl radical having 3 to 10 carbon atoms, an aryl radical having 6 to 12 carbon atoms, an arylalkyl radical having 7 to 12 carbon atoms, a radical of the formula CₘH₂ₘ₊₁(O—CₙH₂ₙ)ₚ—, CₘH₂ₘ₊₁(OOC—CᵥH₂ᵥ)ₚ— or R⁵—C₆H₄(O—CₙH₂ₙ)ₚ—, wherein m=1 to 10, n=2 to 4, p=1 to 15, v=4 or 5, and R⁵ is an alkyl radical having 1 to 12 carbon atoms, preferably an n-alkyl radical having 4 to 10 carbon atoms, a branched alkyl radical having 4 to 10 carbon atoms, a radical of the formula CₘH₂ₘ₊₁(O—CₙH₂ₙ)ₚ— or CₘH₂ₘ₊₁(OOC—CᵥH₂ᵥ)ₚ—, wherein m=1 to 10, n=2 to 4, p=1 to 15 and v=4 or 5, where different R¹ radicals may be the same or different,
R² is

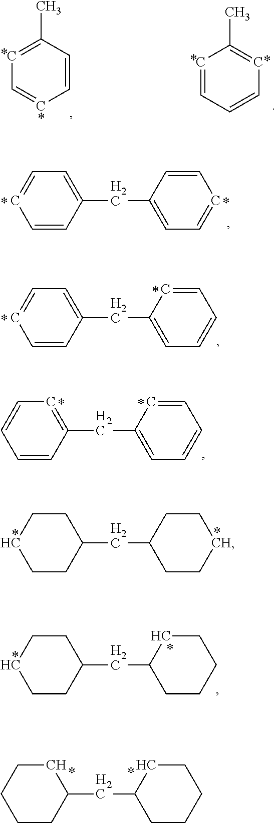

or —(CH₂)_{w}—, wherein w=2 to 10, preferably

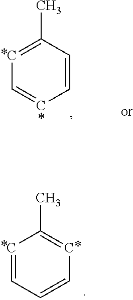

where different R² radicals may the same or different,
R³ is

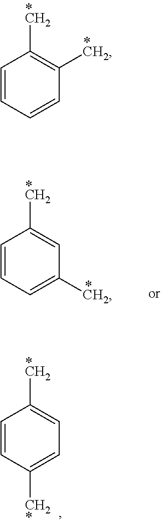

where different R³ radicals may the same or different, and
x is an integer from 1 to 20, preferably 1 to 10.

In the synthesis of the urethane-urea compounds (C), an alcohol is advantageously first reacted with a diisocyanate to give a monoadduct.

R¹—OH+OCN—R²—NCO→R¹—O—C(═O)—NH—R²—NCO

The reaction is preferably effected in the absence of solvents. In order to arrive at the monoisocyanate adduct to a maximum degree, it is advantageous to work with a 1.5- to 5-fold excess of diisocyanate, which can be distilled off again on completion of reaction.

The monoadduct is then reacted with a diamine to give urethane-urea compounds (C).

2R¹—O—C(═O)—NH—R²—NCO+H₂N—R³—NH₂→

R¹—O—C(═O)—NH—R²—NH—C(═O)—NH—R³—NH—C(═O)—NH—R²—NH—C(═O)—OR¹

If excess diisocyanate is still present, what are obtained are urethane-urea compounds of higher molecular weight.

2R¹—O—C(═O)—NH—R²—NCO+(x−1)OCN—R²—NCO+xH₂N—R³—NH₂→

R¹—O—C(═O)—NH—R²—NH—C(═O)[—NH—R³—NH—C(═O)—NH—R²—NH—C(═O)]ₓ—OR¹

This reaction is preferably effected in aprotic solvents, preferably in DMSO, and can advantageously be conducted in the presence of lithium salts, preferably lithium chloride. The proportion of the urethane-urea compounds in the solution is preferably 10% to 75% by weight, preferably 40% to 60% by weight.

In a preferred embodiment, the dental etching composition additionally comprises one or more water-miscible solvents (D).

The water-miscible solvents (D) are preferably selected from the group consisting of ethanol, propan-1-ol, propan-2-ol, butan-1-ol, butan-2-ol, 2-methylpropan-1-ol, 2-methylpropan-2-ol, glycerol, ethylene glycol, propylene glycol, butylene glycol, diethylene glycol, polyethylene glycol, polypropylene glycol, 2-butoxyethan-1-ol, DMSO and acetone, and preferably selected from the group consisting of ethanol, propan-1-ol, propan-2-ol, glycerol, polyethylene glycol, polypropylene glycol and DMSO.

In a preferred embodiment, the dental etching composition additionally comprises colorants (E).

The colorants (E) are preferably selected from the group consisting of dyes, organic color pigments and inorganic color pigments, preferably from dyes, more preferably from phenothiazine dyes,

and/or
the colorants (E) are blue, green or red, preferably blue, colorants.

In a preferred embodiment, the dental etching composition comprises
A) the acid in an amount of 10% to 45% by weight, preferably of 30% to 42% by weight,
B) water in an amount of 30% to 60% by weight, preferably of 40% to 60% by weight,
C) the one or more urethane-urea compounds in an amount of 5% to 20% by weight, preferably of 5% to 15% by weight,
D) water miscible solvent(s) in an amount of 0% to 20% by weight, preferably of 1% to 15% by weight, and
E) colorants in an amount of 0% to 5% by weight, preferably of 0.0001% to 1% by weight,
based in each case on the overall composition.

Since the presence of inorganic solids, especially silica as in the prior art, has the disadvantages described above, the dental etching composition in a particular embodiment is essentially free of fumed silica, preferably essentially free of silica particles, more preferably essentially free of inorganic solids.

What is meant by essentially free is that, within the scope of the industrial preparation options, the content of fumed silica, silica particles or inorganic solids is so low that the adverse effects described do not occur. What is preferably meant by essentially free is therefore a content of fumed silica, silica particles or inorganic solids of less than 1% by weight, preferably less than 0.5% by weight, more preferably less than 0.1% by weight, based in each case on the overall composition. Very particular preference is given to compositions containing no fumed silica, silica particles or inorganic solids at all. Inorganic solids are not considered to include dissolved inorganic substances, especially the lithium salts used in the synthesis of the urethane-urea compounds.

In a preferred embodiment, the dental etching composition does not contain any further constituents apart from constituents (A), (B), (C), (D) and (E).

The dental etching compositions according to the invention are notable for their gel character and for their optimal viscosity for application. More particularly, the dental etching compositions have a loss factor tan δ of less than 1 and/or a viscosity in the range from 0.1 to 200 Pa*s, preferably from 0.5 to 150 Pa*s, more preferably from 1 to 100 Pa*s, most preferably from 1 to 50 Pa*s.

The values of tan δ and viscosity are based on the test method detailed in the description further down and are applicable both to the evaluation point at the end of phase I and to the evaluation point at the end of phase IV.

The dental etching compositions of the invention are additionally also notable for their good storage stability. Thus, the gel character and the optimal viscosity for application are largely maintained even during storage. More particularly, the dental etching compositions preferably, even after storage at 23° C. for 6 months, have a loss factor tan δ of less than 1 and/or a viscosity in the range of 0.1 to 200 Pa*s, preferably of 0.5 to 150 Pa*s, more preferably of 1 to 100 Pa*s, most preferably of 1 to 50 Pa*s.

More preferably, the dental etching compositions, even after storage at 23° C. for 12 months, preferably at 23° C. to 18 months, more preferably at 23° C. for 24 months, have a loss factor tan δ of less than 1 and/or a viscosity in the range of 0.1 to 200 Pa*s, preferably of 0.5 to 150 Pa*s, more preferably of 1 to 100 Pa*s, most preferably of 1 to 50 Pa*s,

and/or
after storage at 37° C. for 6 months, preferably at 37° C. for 12 months, have a loss factor tan δ of less than 1 and/or a viscosity in the range of 0.1 to 200 Pa*s, preferably of 0.5 to 150 Pa*s, more preferably of 1 to 100 Pa*s, most preferably of 1 to 50 Pa*s,
and/or
after storage at 23° C. for 12 months, preferably at 23° C. for 18 months, more preferably at 23° C. for 24 months, have a viscosity that varies from the viscosity prior to storage by not more than ±50%, preferably by not more than ±35%, more preferably by not more than ±20%.

A further aspect of the present invention is the use of a dental etching composition as described above for etching of the hard substance of the tooth.

In a preferred embodiment, this use comprises the steps of
i) optionally desiccating the teeth (or tooth) to be treated, preferably with a dental dam,
ii) applying the dental etching composition to the hard substance of the tooth to be treated,
iii) allowing a contact time of the dental etching composition to achieve an etching effect on the hard substance of the tooth,
iv) rinsing off the dental etching composition,
v) applying a dental primer composition and/or adhesive composition to the etched hard substance of the tooth,
vi) optionally polymerizing the dental primer composition and/or adhesive composition,
vii) applying a dental restoration composition and
viii) polymerizing the dental restoration composition.

The above elucidations relating to the preferred dental etching compositions are likewise applicable to the use thereof.

A further aspect of the present invention is a dental etching composition as described above for use in a therapeutic method of etching the hard substance of the tooth in the course of filling treatment.

In a preferred embodiment, this is a dental etching composition as described above for use in a therapeutic method comprising the steps of
i) optionally desiccating the teeth (or tooth) to be treated, preferably with a dental dam,
ii) applying a dental etching composition as described herein to the hard substance of the tooth to be treated,
iii) allowing a contact time of the dental etching composition to achieve an etching effect on the hard substance of the tooth,
iv) rinsing off the dental etching composition,
v) applying a dental primer composition and/or adhesive composition to the etched hard substance of the tooth,
vi) optionally polymerizing the dental primer composition and/or adhesive composition,
vii) applying a dental restoration composition and
viii) polymerizing the dental restoration composition.

The above elucidations relating to the preferred dental etching compositions are likewise applicable to use thereof in a therapeutic method.

A further aspect of the present invention is a kit comprising

a dental etching composition as described above,

a dental primer composition and/or adhesive composition and

optionally a dental restoration composition.

The above elucidations relating to the preferred dental etching compositions are likewise applicable to a kit comprising said etching composition.

Where particular configurations are described as preferred for any aspect of the invention (composition; use; use in a therapeutic method or kit), the corresponding details are respectively also applicable to the other aspects of the present invention, mutatis mutandis. Preferred individual features of aspects of the invention (as defined in the claims and/or disclosed in the description) are combinable with one another and are preferably combined with one another unless the opposite is apparent to the person skilled in the art from the present text in the individual case.

### EXAMPLES

### Example 1A

To 1.5 mol (261.3 g) of tolylene 2,4-diisocyanate was slowly added dropwise, over the course of 2 hours, 0.5 mol (37.1 g) of 1-butanol. During this addition, the temperature was kept between 50 and 55° C. After the addition had ended, stirring was continued at 50 to 55° C. for a further 3 hours until the theoretical NCO content of 35.2% had been attained. The excess of the diisocyanate was distilled off under reduced pressure (0.1 mbar) at 150 to 170° C. The NCO content was 16.9%, the free TDI content <0.5%.

### Example 1B

To 1.5 mol (261.3 g) of tolylene 2,4-diisocyanate was slowly added dropwise, over the course of 2 hours, 0.5 mol (103.1 g) of triethylene glycol mono-n-butyl ether. During this addition, the temperature was kept between 50 and 55° C. After the addition had ended, stirring was continued at 50 to 55° C. for a further 3 hours until the theoretical NCO content of 28.8% had been attained. The excess of the diisocyanate was distilled off under reduced pressure (0.1 mbar) at 150 to 170° C. The NCO content was 11.0%, the free TDI content <0.5%.

### Example 1C

To 1.5 mol (261.3 g) of tolylene 2,4-diisocyanate was slowly added dropwise, over the course of 2 hours, 0.5 mol (175.0 g) of methoxy polyethylene glycol (MW 350). During this addition, the temperature was kept between 50 and 55° C. After the addition had ended, stirring was continued at 50 to 55° C. for a further 3 hours until the theoretical NCO content of 24.1% had been attained. The excess of the diisocyanate was distilled off under reduced pressure (0.1 mbar) at 150 to 170° C. The NCO content was 8.0%, the free TDI content <0.5%.

### Example 1D

To 1.5 mol (261.3 g) of tolylene 2,4-diisocyanate was slowly added dropwise, over the course of 2 hours, 0.5 mol (275.0 g) of methoxy polyethylene glycol (MW 550). During this addition, the temperature was kept between 50 and 55° C. After the addition had ended, stirring was continued at 50 to 55° C. for a further 3 hours until the theoretical NCO content of 19.6% had been attained. The excess of the diisocyanate was distilled off under reduced pressure (0.1 mbar) at 150 to 170° C. The NCO content was 5.8%, the free TDI content <0.5%.

### Example 1E

To 2.0 mol (348.1 g) of tolylene 2,4-diisocyanate was slowly added dropwise, over the course of 2 hours, 0.5 mol (175.0 g) of methoxy polyethylene glycol (MW 350). During this addition, the temperature was kept between 50 and 55° C. After the addition had ended, stirring was continued at 50 to 55° C. for a further 3 hours until the theoretical NCO content of 28.1% had been attained. The excess of the diisocyanate was distilled off under reduced pressure (0.1 mbar) at 150 to 170° C. The NCO content was 8.0%, the free TDI content <0.5%.

### Example 1F

To 1.0 mol (174.2 g) of tolylene 2,4-diisocyanate was slowly added dropwise, over the course of 2 hours, 0.5 mol (175.0 g) of methoxy polyethylene glycol (MW 350). During this addition, the temperature was kept between 50 and 55° C. After the addition had ended, stirring was continued at 50 to 55° C. for a further 3 hours until the theoretical NCO content of 18.0% had been attained. The excess of the diisocyanate was distilled off under reduced pressure (0.1 mbar) at 150 to 170° C. The NCO content was 8.0%, the free TDI content <0.5%.

### Example 1G

To 1.5 mol (375.4 g) of diphenylmethane 4,4′-diisocyanate was slowly added dropwise, over the course of 2 hours, 0.5 mol (37.1 g) of 1-butanol. During this addition, the temperature was kept between 50 and 55° C. After the addition had ended, stirring was continued at 50 to 55° C. for a further 3 hours until the theoretical NCO content of 25.5% had been attained. The excess of the diisocyanate was distilled off under reduced pressure (0.1 mbar) at 150 to 170° C.

The NCO content was 13.0%, the free MDI content <0.5%.

### Example 1H

To 1.5 mol (252.3 g) of hexamethylene 1,6-diisocyanate was slowly added dropwise, over the course of 2 hours, 0.5 mol (37.1 g) of 1-butanol. During this addition, the temperature was kept between 50 and 55° C. After the addition had ended, stirring was continued at 50 to 55° C. for a further 3 hours until the theoretical NCO content of 36.3% had been attained. The excess of the diisocyanate was distilled off under reduced pressure (0.1 mbar) at 150 to 170° C. The NCO content was 17.3%, the free HMDI content <0.5%.

TABLE 1A
Examples 1A to 1D

| Example | 1A | 1B | 1C | 1D |
|---|---|---|---|---|
| Diisocyanate | Tolylene | Tolylene | Tolylene | Tolylene |
| | 2,4- | 2,4- | 2,4- | 2,4- |
| | diisocyanate | diisocyanate | diisocyanate | diisocyanate |
| Alcohol | 1-Butanol | Triethylene | Methoxy | Methoxy |
| | | glycol mono- | polyethylene | polyethylene |
| | | butyl ether | glycol 350 | glycol 550 |
| Diisocyanate/ | 3:1 | 3:1 | 3:1 | 3:1 |
| alcohol ratio | | | | |
| NCO content | 16.9% | 11.0% | 8.0% | 5.8% |

TABLE 1B
Examples 1E to 1H

| Example | 1E | 1F | 1G | 1H |
|---|---|---|---|---|
| Diisocyanate | Tolylene | Tolylene | Diphenyl- | Hexa- |
| | 2,4- | 2,4- | methane | methylene |
| | diisocyanate | diisocyanate | 4,4′- | 1,6- |
| | | | diisocyanate | diisocyanate |
| Alcohol | Methoxy | Methoxy | 1-Butanol | 1-Butanol |
| | polyethylene | polyethylene | | |
| | glycol 350 | glycol 350 | | |
| Diisocyanate/ | 4:1 | 2:1 | 3:1 | 3:1 |
| alcohol ratio | | | | |
| NCO content | 8.0% | 8.0% | 13.0% | 17.3% |

### Example 2A

17.0 g (0.4 mol) of lithium chloride and 34.1 g (0.25 mol) of xylylene-1,3-diamine were dissolved in 175 g of DMSO at 80° C. Subsequently, over the course of one hour, 124.2 g of example 1A was added. On completion of addition, the mixture was stirred at 80° C. for a further 30 minutes and then cooled down to room temperature. The proportion of dissolved solids in the resultant urethane-urea solution was 50%.

### Example 2B

17.0 g (0.4 mol) of lithium chloride and 34.1 g (0.25 mol) of xylylene-1,3-diamine were dissolved in 224 g of DMSO at 80° C. Subsequently, over the course of one hour, 190.2 g of example 1B was added. On completion of addition, the mixture was stirred at 80° C. for a further 30 minutes and then cooled down to room temperature. The proportion of dissolved solids in the resultant urethane-urea solution was 50%.

### Example 2C

17.0 g (0.4 mol) of lithium chloride and 34.1 g (0.25 mol) of xylylene-1,3-diamine were dissolved in 313 g of DMSO at 80° C. Subsequently, over the course of one hour, 262.1 g of example 1C was added. On completion of addition, the mixture was stirred at 80° C. for a further 30 minutes and then cooled down to room temperature. The proportion of dissolved solids in the resultant urethane-urea solution was 50%.

### Example 2D

17.0 g (0.4 mol) of lithium chloride and 34.1 g (0.25 mol) of xylylene-1,3-diamine were dissolved in 413 g of DMSO at 80° C. Subsequently, over the course of one hour, 362.1 g of example 1D was added. On completion of addition, the mixture was stirred at 80° C. for a further 30 minutes and then cooled down to room temperature. The proportion of dissolved solids in the resultant urethane-urea solution was 50%.

### Example 2E

17.0 g (0.4 mol) of lithium chloride and 34.1 g (0.25 mol) of xylylene-1,3-diamine were dissolved in 313 g of DMSO at 80° C. Subsequently, over the course of one hour, 262.1 g of example 1E was added. On completion of addition, the mixture was stirred at 80° C. for a further 30 minutes and then cooled down to room temperature. The proportion of dissolved solids in the resultant urethane-urea solution was 50%.

### Example 2F

17.0 g (0.4 mol) of lithium chloride and 34.1 g (0.25 mol) of xylylene-1,3-diamine were dissolved in 313 g of DMSO at 80° C. Subsequently, over the course of one hour, 262.1 g of example 1F was added. On completion of addition, the mixture was stirred at 80° C. for a further 30 minutes and then cooled down to room temperature. The proportion of dissolved solids in the resultant urethane-urea solution was 50%.

### Example 2G

17.0 g (0.4 mol) of lithium chloride and 34.1 g (0.25 mol) of xylylene-1,3-diamine were dissolved in 213 g of DMSO at 80° C. Subsequently, over the course of one hour, 162.2 g of example 1G was added. On completion of addition, the mixture was stirred at 80° C. for a further 30 minutes and then cooled down to room temperature. The proportion of dissolved solids in the resultant urethane-urea solution was 50%.

### Example 2H

17.0 g (0.4 mol) of lithium chloride and 34.1 g (0.25 mol) of xylylene-1,3-diamine were dissolved in 172 g of DMSO at 80° C. Subsequently, over the course of one hour, 121.2 g of example 1H was added. On completion of addition, the mixture was stirred at 80° C. for a further 30 minutes and then cooled down to room temperature. The proportion of dissolved solids in the resultant urethane-urea solution was 50%.

### Example 2I

17.0 g (0.4 mol) of lithium chloride and 34.1 g (0.25 mol) of xylylene-1,2-diamine were dissolved in 313 g of DMSO at 80° C. Subsequently, over the course of one hour, 262.1 g of example 1C was added. On completion of addition, the mixture was stirred at 80° C. for a further 30 minutes and then cooled down to room temperature. The proportion of dissolved solids in the resultant urethane-urea solution was 50%.

### Example 2J

17.0 g (0.4 mol) of lithium chloride and 34.1 g (0.25 mol) of xylylene-1,4-diamine were dissolved in 313 g of DMSO at 80° C. Subsequently, over the course of one hour, 262.1 g of example 1C was added. On completion of addition, the mixture was stirred at 80° C. for a further 30 minutes and then cooled down to room temperature. The proportion of dissolved solids in the resultant urethane-urea solution was 50%.

### Example 2K

17.0 g (0.4 mol) of lithium chloride and 35.6 g (0.25 mol) of 1,3-bis(aminomethyl)cyclohexane were dissolved in 315 g of DMSO at 80° C. Subsequently, over the course of one hour, 262.1 g of example 1C was added. On completion of addition, the mixture was stirred at 80° C. for a further 30 minutes and then cooled down to room temperature. The proportion of dissolved solids in the resultant urethane-urea solution was 50%.

### Example 2L

27.6 g (0.4 mol) of lithium nitrate and 34.1 g (0.25 mol) of xylylene-1,3-diamine were dissolved in 324 g of DMSO at 80° C. Subsequently, over the course of one hour, 262.1 g of example 1C was added. On completion of addition, the mixture was stirred at 80° C. for a further 30 minutes and then cooled down to room temperature. The proportion of dissolved solids in the resultant urethane-urea solution was 50%.

### Example 2M

17.0 g (0.4 mol) of lithium chloride and 34.1 g (0.25 mol) of xylylene-1,3-diamine were dissolved in 209 g of DMSO at 80° C. Subsequently, over the course of one hour, 262.1 g of example 1C was added. On completion of addition, the mixture was stirred at 80° C. for a further 30 minutes and then cooled down to room temperature. The proportion of dissolved solids in the resultant urethane-urea solution was 40%.

### Example 2N

17.0 g (0.4 mol) of lithium chloride and 34.1 g (0.25 mol) of xylylene-1,3-diamine were dissolved in 470 g of DMSO at 80° C. Subsequently, over the course of one hour, 262.1 g of example 1C was added. On completion of addition, the mixture was stirred at 80° C. for a further 30 minutes and then cooled down to room temperature. The proportion of dissolved solids in the resultant urethane-urea solution was 60%.

TABLE 2A
Examples 2A to 2D

| Example | 2A | 2B | 2C | 2D |
|---|---|---|---|---|
| Monoadduct | 1A | 1B | 1C | 1D |
| Diamine | Xylylene- | Xylylene- | Xylylene- | Xylylene- |
| | 1,3- | 1,3- | 1,3- | 1,3- |
| | diamine | diamine | diamine | diamine |
| Li salt | LiCl | LiCl | LiCl | LiCl |
| Solids content | 50% | 50% | 50% | 50% |

TABLE 2B
Examples 2E to 2H

| Example | 2E | 2F | 2G | 2H |
|---|---|---|---|---|
| Monoadduct | 1E | 1F | 1G | 1H |
| Diamine | Xylylene- | Xylylene- | Xylylene- | Xylylene- |
| | 1,3- | 1,3- | 1,3- | 1,3- |
| | diamine | diamine | diamine | diamine |
| Li salt | LiCl | LiCl | LiCl | LiCl |
| Solids content | 50% | 50% | 50% | 50% |

TABLE 2C
Examples 21 to 2L

| Example | 2I | 2J | 2K | 2L |
|---|---|---|---|---|
| Monoadduct | 1C | 1C | 1C | 1C |
| Diamine | Xylylene- | Xylylene- | 1,3-Bis(amino- | Xylylene- |
| | 1,2- | 1,4- | methyl)cyclo- | 1,3- |
| | diamin | diamin | hexane | diamine |
| Li salt | LiCl | LiCl | LiCl | LiNO₃ |
| Solids content | 50% | 50% | 50% | 50% |

TABLE 2D
Examples 2M to 2N

| Example | 2M | 2N |
|---|---|---|
| Monoadduct | 1C | 1C |
| Diamine | Xylylene- | Xylylene- |
| | 1,3- | 1,3- |
| | diamine | diamine |
| Li salt | LiCl | LiCl |
| Solids content | 40% | 60% |

### Example 3A

In a beaker, 41.2 g of 85% phosphoric acid and 1.0 g of PEG-400 and 0.01 g of methylene blue were dissolved in 39.4 g of demineralized water while stirring. Subsequently, 18.4 g of the DMSO solution from example 2A was added in portions while stirring and the mixture was stirred at room temperature for a further 30 minutes.

### Examples 3B to 3N

Analogously to example 3A, etching gels 3B to 3N were produced using, rather than the DMSO solution from example 2A, the DMSO solutions from examples 2B to 2N.

### Example 3O

In a beaker, 41.2 g of 85% phosphoric acid and 1.0 g of glycerol and 0.01 g of methylene blue were dissolved in 39.4 g of demineralized water while stirring. Subsequently, 18.4 g of the DMSO solution from example 2A was added in portions while stirring and the mixture was stirred at room temperature for a further 30 minutes.

### Example 3P

In a beaker, 41.2 g of 85% phosphoric acid and 2.0 g of ethanol and 0.01 g of methylene blue were dissolved in 38.4 g of demineralized water while stirring. Subsequently, 18.4 g of the DMSO solution from example 2A was added in portions while stirring and the mixture was stirred at room temperature for a further 30 minutes.

### Example 3Q

In a beaker, 41.2 g of 85% phosphoric acid and 0.01 g of methylene blue were dissolved in 40.4 g of demineralized water while stirring. Subsequently, 18.4 g of the DMSO solution from example 2A was added in portions while stirring and the mixture was stirred at room temperature for a further 30 minutes.

### Example 3R

In a beaker, 41.2 g of 85% phosphoric acid, 2.5 g of PEG-400, 4.5 g of glycerol and 0.01 g of methylene blue were dissolved in 39.4 g of demineralized water while stirring. Subsequently, 12.4 g of the DMSO solution from example 2A was added in portions while stirring and the mixture was stirred at room temperature for a further 30 minutes.

### Example 3S

In a beaker, 41.2 g of 85% phosphoric acid, 1.0 g of PEG-400 and 1.5 g of glycerol and 0.01 g of methylene blue were dissolved in 41.3 g of demineralized water while stirring. Subsequently, 15.0 g of the DMSO solution from example 2A was added in portions while stirring and the mixture was stirred at room temperature for a further 30 minutes.

### Example 3T

In a beaker, 41.2 g of 85% phosphoric acid and 1.0 g of glycerol and 0.01 g of methylene blue were dissolved in 36.8 g of demineralized water while stirring. Subsequently, 22.0 g of the DMSO solution from example 2A was added in portions while stirring and the mixture was stirred at room temperature for a further 30 minutes.

### Example 3U

In a beaker, 41.2 g of 85% phosphoric acid and 0.5 g of glycerol and 0.01 g of methylene blue were dissolved in 33.9 g of demineralized water while stirring. Subsequently, 24.4 g of the DMSO solution from example 2A was added in portions while stirring and the mixture was stirred at room temperature for a further 30 minutes.

TABLE 3A
Examples 3A to 3Q

| | | 3A to 3N | 3O | 3P | 3Q |
|---|---|---|---|---|---|
| (A)*¹ | H₃PO₄*¹ | 35.02 | 35.02 | 35.02 | 35.02 |
| (B)*² | Water*² | 45.57 | 45.57 | 44.57 | 46.57 |
| (C)*³ | Urethane-urea | 9.20 | 9.20 | 9.20 | 9.20 |
| | compound*³ | | | | |
| (D)*⁴ | PEG-400 | 1.00 | | | |
| | Glycerol | | 1.00 | | |
| | Ethanol | | | 2.00 | |
| | DMSO*⁴ | 9.20 | 9.20 | 9.20 | 9.20 |
| (E) | Methylene blue | 0.01 | 0.01 | 0.01 | 0.01 |
| Total | | 100.00 | 100.00 | 100.00 | 100.00 |

*¹Since 85% phosphoric acid was used in the examples, only the actual proportion of phosphoric acid is stated here under (A).
*²As well as the water used, the water content from the phosphoric acid is also stated under (B).
*³Since Examples 2 are a solution of the urethane-urea compound, only the actual proportion of the urethane-urea compound is stated under (C).
*⁴As well as any further water-miscible solvents, the proportion of the solvent in the solution of the urethane-urea compound is also stated under (D).

TABLE 3B
Examples 3R to 3U

| | | 3R | 3S | 3T | 3U |
|---|---|---|---|---|---|
| (A)*¹ | H₃PO₄*¹ | 35.02 | 35.02 | 35.02 | 35.02 |
| (B)*² | Water*² | 45.57 | 47.47 | 41.97 | 40.07 |
| (C)*³ | Urethane-urea | 6.20 | 7.50 | 11.00 | 12.20 |
| | compound*³ | | | | |
| (D)*⁴ | PEG-400 | 2.50 | 1.00 | | |
| | Glycerol | 4.50 | 1.50 | 1.00 | 0.50 |
| | DM SO*⁴ | 6.20 | 7.50 | 11.00 | 12.20 |
| (E) | Methylene blue | 0.01 | 0.01 | 0.01 | 0.01 |
| Total | | 100.00 | 100.00 | 100.00 | 100.00 |

*¹Since 85% phosphoric acid was used in the examples, only the actual proportion of phosphoric acid is stated here under (A).
*²As well as the water used, the water content from the phosphoric acid is also stated under (B).
*³Since Examples 2 are a solution of the urethane-urea compound, only the actual proportion of the urethane-urea compound is stated under (C).
*⁴As well as any further water-miscible solvents, the proportion of the solvent in the solution of the urethane-urea compound is also stated under (D).

### Comparative Example 4A

In a beaker, 10.0 g of gum arabic and 0.01 g of methylene blue were dissolved in 48.8 g of demineralized water while stirring. Subsequently, 41.2 g of 85% phosphoric acid was added while stirring and the mixture was stirred at room temperature for a further 30 minutes. No thickening effect occurred. The solution was of low viscosity.

### Comparative Example 4B

In a beaker, 20.0 g of gum arabic and 0.01 g of methylene blue were dissolved in 38.8 g of demineralized water while stirring. Subsequently, 41.2 g of 85% phosphoric acid was added while stirring and the mixture was stirred at room temperature for a further 30 minutes. No thickening effect occurred. The solution was of low viscosity.

### Comparative Example 4C

In a beaker, 2.0 g of xanthan gum and 0.01 g of methylene blue were dissolved in 56.8 g of demineralized water while stirring. Subsequently, 41.2 g of 85% phosphoric acid was added while stirring and the mixture was stirred at room temperature for a further 30 minutes. The viscosity was 3.0 Pa*s. During storage at 23° C., there was a gradual rise in viscosity over the course of 6 months to 4.2 Pa*s. Even after a storage time of one month at 23° C., distinct formation of gas occurred.

### Comparative Example 4D

In a beaker, 5.0 g of xanthan gum and 0.01 g of methylene blue were dissolved in 53.8 g of demineralized water while stirring. Subsequently, 41.2 g of 85% phosphoric acid was added while stirring and the mixture was stirred at room temperature for a further 30 minutes. The viscosity was 6.0 Pa*s. During storage at 23° C., there was a gradual rise in viscosity over the course of 6 months to 8.3 Pa*s. Even after a storage time of one month at 23° C., distinct formation of gas occurred.

### Comparative Example 4E

In a beaker, 10.0 g of polyvinylalcohol and 0.01 g of methylene blue were dissolved in 48.8 g of demineralized water while stirring. Subsequently, 41.2 g of 85% phosphoric acid was added while stirring and the mixture was stirred at room temperature for a further 30 minutes. Barely any thickening effect occurred. The solution was of low viscosity.

### Comparative Example 4F

In a beaker, 20.0 g of polyvinylalcohol and 0.01 g of methylene blue were dissolved in 38.8 g of demineralized water while stirring. Subsequently, 41.2 g of 85% phosphoric acid was added while stirring and the mixture was stirred at room temperature for a further 30 minutes. Barely any thickening effect occurred. The solution was of low viscosity.

### Comparative Example 4G

In a beaker, 30.0 g of polyvinylalcohol and 0.01 g of methylene blue were dissolved in 28.8 g of demineralized water while stirring. Subsequently, 41.2 g of 85% phosphoric acid was added while stirring and the mixture was stirred at room temperature for a further 30 minutes. The viscosity was 10.0 Pa*s. The material did not have good applicability to the prepared tooth surface. It become fluid as a result of the movement on application and flowed off the tooth.

### Comparative Example 4H

In a beaker, 2.5 g of hydroxyethyl cellulose and 0.01 g of methylene blue were dissolved in 56.3 g of demineralized water while stirring. Subsequently, 41.2 g of 85% phosphoric acid was added while stirring and the mixture was stirred at room temperature for a further 30 minutes. Only a minor thickening effect occurred. The solution was of relatively low viscosity.

### Comparative Example 4I

In a beaker, 5.0 g of hydroxyethyl cellulose and 0.01 g of methylene blue were dissolved in 53.8 g of demineralized water while stirring. Subsequently, 41.2 g of 85% phosphoric acid was added while stirring and the mixture was stirred at room temperature for a further 30 minutes. The viscosity was 6.5 Pa*s. During storage at 23° C., the viscosity already decreased to 2.5 Pa*s within one month.

### Comparative Example 4J

In a beaker, 20.0 g of glycerol and 0.01 g of methylene blue were dissolved in 38.8 g of demineralized water while stirring. Subsequently, 41.2 g of 85% phosphoric acid was added while stirring and the mixture was stirred at room temperature for a further 30 minutes. No thickening effect occurred. The solution was of low viscosity.

### Comparative Example 4K

In a beaker, 40.0 g of glycerol and 0.01 g of methylene blue were dissolved in 18.8 g of demineralized water while stirring. Subsequently, 41.2 g of 85% phosphoric acid was added while stirring and the mixture was stirred at room temperature for a further 30 minutes. No thickening effect occurred. The solution was of low viscosity.

### Comparative Example 4L

In a beaker, 0.01 g of methylene blue was dissolved in 58.8 g of glycerol while stirring. Subsequently, 41.2 g of 85% phosphoric acid was added while stirring and the mixture was stirred at room temperature for a further 30 minutes. No thickening effect occurred. The solution was of low viscosity.

### Comparative Example 4M

In a beaker, 2.5 g of carboxymethyl cellulose and 0.01 g of methylene blue were dissolved in 56.3 g of demineralized water while stirring. Subsequently, 41.2 g of 85% phosphoric acid was added while stirring and the mixture was stirred at room temperature for a further 30 minutes. The viscosity was 2.5 Pa*s, but decreased noticeably during storage, and the gel become more fluid.

### Comparative Example 4N

In a beaker, 5.0 g of carboxymethyl cellulose and 0.01 g of methylene blue were dissolved in 53.8 g of demineralized water while stirring. Subsequently, 41.2 g of 85% phosphoric acid was added while stirring and the mixture was stirred at room temperature for a further 30 minutes. The viscosity was 8.2 Pa*s, but decreased noticeably during storage, and the gel become more fluid.

### Comparative Example 4O

In a beaker, 0.01 g of methylene blue was dissolved in 58.8 g of demineralized water while stirring. Subsequently, 41.2 g of 85% phosphoric acid was added while stirring. Subsequently, 5.0 g of Aerosil A200 was added and dispersed with an Ultra Turrax for 5 minutes. The viscosity was 10.4 Pa*s, but increased noticeably during storage, and the gel thickened.

### Comparative Example 4P

In a beaker, 0.01 g of methylene blue was dissolved in 8.8 g of demineralized water while stirring. Subsequently, first 50.0 g of Snowtex ST-O (20% colloidal silica in water; particle size 10-20 nm) and then 41.2 g of 85% phosphoric acid were added while stirring and the mixture was stirred at room temperature for a further 30 minutes. The viscosity was 8.8 Pa*s, but increased noticeably during storage, and the gel thickened.

TABLE 4A
Comparative Examples 4A to 4D

| | | 4A | 4B | 4C | 4D |
|---|---|---|---|---|---|
| (A)*¹ | H₃PO₄*¹ | 35.02 | 35.02 | 35.02 | 35.02 |
| (B)*² | Water*² | 54.97 | 44.97 | 62.97 | 59.97 |
| (X) | Gum arabic | 10.00 | 20.00 | | |
| | Xanthan gum | | | 2.00 | 5.00 |
| (E) | Methylene blue | 0.01 | 0.01 | 0.01 | 0.01 |
| Total | | 100.00 | 100.00 | 100.00 | 100.00 |

TABLE 4B
Comparative Examples 4E to 4H

| | | 4E | 4F | 4G | 4H |
|---|---|---|---|---|---|
| (A)*¹ | H₃PO₄*¹ | 35.02 | 35.02 | 35.02 | 35.02 |
| (B)*² | Water*² | 54.97 | 44.97 | 34.97 | 62.47 |
| (X) | Polyvinylalcohol | 10.00 | 20.00 | 30.00 | |
| | Hydroxyethyl | | | | 2.50 |
| | cellulose | | | | |
| (E) | Methylene blue | 0.01 | 0.01 | 0.01 | 0.01 |
| Total | | 100.00 | 100.00 | 100.00 | 100.00 |

TABLE 4C
Comparative Examples 4I to 4L

| | | 4I | 4J | 4K | 4L |
|---|---|---|---|---|---|
| (A)*¹ | H₃PO₄*¹ | 35.02 | 35.02 | 35.02 | 35.02 |
| (B)*² | Water*² | 59.97 | 44.97 | 24.97 | 6.18 |
| (X) | Hydroxyethyl | 5.00 | | | |
| | cellulose | | | | |
| (D) | Glycerol | | 20.00 | 40.00 | 58.79 |
| (E) | Methylene blue | 0.01 | 0.01 | 0.01 | 0.01 |
| Total | | 100.00 | 100.00 | 100.00 | 100.00 |

TABLE 4D
Comparative Examples 4M to 4P

| | | 4M | 4N | 4O | 4P |
|---|---|---|---|---|---|
| (A)*¹ | H₃PO₄*¹ | 35.02 | 35.02 | 35.02 | 35.02 |
| (B)*^{2, 5} | Water*^{2, 5} | 62.47 | 59.97 | 59.97 | 54.97 |
| (X) | Carboxymethyl | 2.50 | 5.00 | | |
| | cellulose | | | | |
| | Fumed silica | | | 5.00 | |
| | Colloidal silica*⁶ | | | | 10.00 |
| (E) | Methylene blue | 0.01 | 0.01 | 0.01 | 0.01 |
| Total | | 100.00 | 100.00 | 100.00 | 100.00 |

*¹ Since 85% phosphoric acid was used in the examples, only the actual proportion of phosphoric acid is stated here under (A).
*² As well as the water used, the water content from the phosphoric acid is also stated under (B).
*⁵ When the aqueous dispersion of colloidal silica is used, the water content from the dispersion is also stated under (B).
*⁶ For the colloidal silica, only the SiO₂ content is stated under (X).

### Shear Bond Strength:

The tests for shear bond strength were conducted in accordance with ISO 29022:2013. Bovine front teeth were embedded in an epoxy matrix in the form of a cylinder having diameter d=2.5 cm, then the enamel or dentine surface was exposed. The surface of the teeth was standardized by coarse grinding with abrasive paper of P120 grit (125±1 μm) and then fine grinding with abrasive paper of P400 grit (35±1 μm). The tooth surface thus prepared was freed of impurities under flowing deionized water and then freed of excess water by a gentle/briefly applied jet of oil- and water-free compressed air immediately prior to the application of the etchant. The teeth must not be overdried in order to prevent morphological changes in the hard substance of the tooth. The etchant was applied directly from the syringe over the area of the hard substance of the tooth and left at rest for 30 s (enamel) or 15 s (dentine). This was followed by rinsing-off under flowing water for several seconds. The etched tooth obtained was freed of excess water by a gentle/briefly applied jet of oil- and water-free compressed air and processed further while moist. The adhesive (Futurabond U, VOCO GmbH) was applied to the prepared tooth surface and massaged into the surface for 20 s. Solvents present in the adhesive were removed by blowing with a jet of oil- and water-free compressed air for 5 seconds. This was followed by curing with light for 10 s (Celalux 2, VOCO GmbH, 420-490 nm, 1000 W/cm²). After curing, the embedded tooth specimen was introduced into a bonding clamp including insert form (in accordance with ISO 29022:2013—from Ultradent Products, South Jordan). The insert form was applied to the surface of the tooth, checked for an adequate fit and fixed to the screws of the apparatus. The composite (GrandioSO A1, VOCO GmbH) was applied to the composite face in the recess of the insert form with a filling instrument and then cured by light for 10 seconds (Celalux 2, VOCO GmbH, 420-490 nm, 1000 W/cm²). The composite specimen was removed from the bonding clamp and stored in water at (37±2) ° C. for (24±2) h. Removal from the water was followed immediately by determination of shear bond strength. For this purpose, the composite test specimens were subjected to stress in a shear test using a universal tester (ZwickRoell GmbH & Co. KG, Ulm) at a crosshead speed of (1.0±0.1) mm/min and an initial force of 1 N until fracture. Shear strength in MPa is found as the quotient of breaking force in N and bonded area in mm².

### Viscosity:

Viscosity was determined with a Physica MCR 301 rheometer (Anton Paar) in an oscillation test (plate/plate) at 23° C. The plate diameter was 50 mm and the plate distance 1 mm. The method of measuring the viscosities comprises four successive phases.

In phase I of the measurement, measurement was effected at a deformation of 0.1% and an oscillation frequency of 10 Hz for five minutes (measurement point duration 5 s). The last point of phase I was used here for evaluation.

In phase II of the measurement, measurement was effected at an oscillation frequency of 10 Hz for 60 s (measurement point duration 1 s), with increasing deformation by 5 percentage points per second from 0.1% to 300%.

In phase III of the measurement, measurement was again effected at a deformation of 0.1% and an oscillation frequency of 10 Hz for 60 s (measurement point duration 1).

In phase IV of the measurement, measurement was continued at a deformation of 0.1% and an oscillation frequency of 10 Hz, but with a greater measurement point duration, for 4 minutes (measurement point duration 4 s). The last point of phase IV was used here for evaluation.

The evaluation point of phase I here describes the viscosity at the state of rest, and the evaluation point of phase IV the viscosity on completion of shear stress (i.e., after application to the surface of the tooth).

TABLE 5A
Examples 3A-3G

| Example | 3A | 3B | 3C | 3D | 3E | 3F | 3G |
|---|---|---|---|---|---|---|---|
| Adhesion (dentine) | 33.2 | 31.8 | 32.2 | 31.7 | 32.5 | 32.3 | 33.3 |
| [MPa] | | | | | | | |
| Adhesion (enamel) | 35.2 | 34.5 | 34.7 | 34.7 | 35.0 | 35.1 | 35.1 |
| [MPa] | | | | | | | |
| Viscosity (I) [Pa*s] | 2.5 | 2.5 | 2.6 | 2.4 | 2.7 | 2.3 | 2.3 |
| Viscosity (IV) [Pa*s] | 2.5 | 2.5 | 2.6 | 2.4 | 2.7 | 2.2 | 2.3 |
| tan δ (I) | 0.89 | 0.83 | 0.85 | 0.82 | 0.90 | 0.90 | 0.83 |
| tan δ (IV) | 0.89 | 0.84 | 0.85 | 0.83 | 0.91 | 0.91 | 0.84 |
| Viscosity (I) (6 | 2.5 | 2.5 | 2.5 | 2.4 | 2.7 | 2.2 | 2.2 |
| months, 23° C.) [Pa*s] | | | | | | | |
| Viscosity (IV) (6 | 2.4 | 2.5 | 2.5 | 2.3 | 2.6 | 2.1 | 2.2 |
| months, 23° C.) [Pa*s] | | | | | | | |
| tan δ (I) (6 months, | 0.90 | 0.89 | 0.86 | 0.87 | 0.85 | 0.93 | 0.90 |
| 23° C.) | | | | | | | |
| tan δ (IV) (6 months, | 0.90 | 0.90 | 0.88 | 0.90 | 0.87 | 0.96 | 0.90 |
| 23° C.) | | | | | | | |

TABLE 5B
Examples 3H-3N

| Example | 3H | 3I | 3J | 3K | 3L | 3M | 3N |
|---|---|---|---|---|---|---|---|
| Adhesion (dentine) | 32.3 | 31.1 | 30.7 | 33.6 | 31.2 | 33.1 | 32.1 |
| [MPa] | | | | | | | |
| Adhesion (enamel) | 35.2 | 35.5 | 34.6 | 34.8 | 35.5 | 34.5 | 34.9 |
| [MPa] | | | | | | | |
| Viscosity (I) [Pa*s] | 2.4 | 2.1 | 2.4 | 2.3 | 2.6 | 2.8 | 2.9 |
| Viscosity (IV) [Pa*s] | 2.4 | 2.0 | 2.3 | 2.3 | 2.5 | 2.7 | 2.8 |
| tan δ (I) | 0.88 | 0.89 | 0.87 | 0.87 | 0.81 | 0.79 | 0.88 |
| tan δ (IV) | 0.90 | 0.90 | 0.89 | 0.88 | 0.83 | 0.81 | 0.90 |
| Viscosity (I) (6 | 2.4 | 2.0 | 2.3 | 2.3 | 2.6 | 2.7 | 2.8 |
| months, 23° C.) [Pa*s] | | | | | | | |
| Viscosity (IV) (6 | 2.3 | 2.0 | 2.3 | 2.2 | 2.5 | 2.6 | 2.7 |
| months, 23° C.) [Pa*s] | | | | | | | |
| tan δ (I) (6 months, | 0.88 | 0.90 | 0.89 | 0.88 | 0.82 | 0.80 | 0.88 |
| 23° C.) | | | | | | | |
| tan δ (IV) (6 months, | 0.89 | 0.91 | 0.91 | 0.89 | 0.85 | 0.81 | 0.89 |
| 23° C.) | | | | | | | |

TABLE 5C
Examples 3O-3U

| Example | 3O | 3P | 3Q | 3R | 3S | 3T | 3U |
|---|---|---|---|---|---|---|---|
| Adhesion (dentine) | 31.3 | 31.6 | 32.7 | 30.1 | 32.4 | 32.1 | 30.0 |
| [MPa] | | | | | | | |
| Adhesion (enamel) | 34.9 | 35.3 | 35.4 | 33.9 | 33.8 | 34.1 | 33.1 |
| [MPa] | | | | | | | |
| Viscosity (I) [Pa*s] | 1.8 | 1.3 | 1.1 | 1.9 | 2.1 | 3.5 | 4.6 |
| Viscosity (IV) [Pa*s] | 1.7 | 1.3 | 1.1 | 1.8 | 2.0 | 3.2 | 4.3 |
| tan δ (I) | 0.91 | 0.97 | 0.99 | 0.90 | 0.87 | 0.75 | 0.91 |
| tan δ (IV) | 0.92 | 0.98 | 0.99 | 0.92 | 0.89 | 0.78 | 0.92 |
| Viscosity (I) (6 | 1.7 | 1.4 | 1.2 | 1.8 | 2.0 | 3.4 | 4.4 |
| months, 23° C.) [Pa*s] | | | | | | | |
| Viscosity (IV) (6 | 1.7 | 1.4 | 1.2 | 1.1 | 1.9 | 3.2 | 4.1 |
| months, 23° C.) [Pa*s] | | | | | | | |
| tan δ (I) (6 months, | 0.91 | 0.95 | 0.96 | 0.90 | 0.86 | 0.79 | 0.91 |
| 23° C.) | | | | | | | |
| tan δ (IV) (6 months, | 0.92 | 0.96 | 0.98 | 0.91 | 0.88 | 0.83 | 0.92 |
| 23° C.) | | | | | | | |

TABLE 6A
Comparative Examples 4A-4G

| Comparative Example | 4A | 4B | 4C | 4D | 4E | 4F | 4G |
|---|---|---|---|---|---|---|---|
| Adhesion (dentine) | 14.2 | 15.1 | 17.3 | 16.8 | 15.3 | 12.8 | 11.5 |
| [MPa] | | | | | | | |
| Adhesion (enamel) | 16.3 | 18.4 | 20.4 | 19.1 | 17.7 | 14.3 | 12.3 |
| [MPa] | | | | | | | |
| Viscosity (I) [Pa*s] | 0.2 | 0.3 | 3.0 | 6.0 | 0.6 | 0.8 | 10.0 |
| Viscosity (IV) [Pa*s] | 0.2 | 0.3 | 2.9 | 5.8 | 0.6 | 0.8 | 1.1 |
| tan δ (I) | 1.50 | 1.39 | 0.88 | 0.82 | 1.28 | 1.22 | 1.50 |
| tan δ (IV) | 1.50 | 1.39 | 0.90 | 0.85 | 1.29 | 1.23 | 1.50 |
| Viscosity (I) (6 | n.d. | n.d. | 4.2 | 8.3 | n.d. | n.d. | n.d. |
| months, 23° C.) [Pa*s] | | | | | | | |
| Viscosity (IV) (6 | n.d. | n.d. | 4.1 | 8.0 | n.d. | n.d. | n.d. |
| months, 23° C.) [Pa*s] | | | | | | | |
| tan δ (I) (6 months, | n.d. | n.d. | 0.81 | 0.75 | n.d. | n.d. | n.d. |
| 23° C.) | | | | | | | |
| tan δ (IV) (6 months, | n.d. | n.d. | 0.84 | 0.79 | n.d. | n.d. | n.d. |
| 23° C.) | | | | | | | |

TABLE 6B
Comparative Examples 4H-4N

| Comparative Example | 4H | 4I | 4J | 4K | 4L | 4M | 4N |
|---|---|---|---|---|---|---|---|
| Adhesion (dentine) | 20.4 | 20.8 | n.d. | n.d. | n.d. | 18.4 | 19.7 |
| [MPa] | | | | | | | |
| Adhesion (enamel) | 22.3 | 22.5 | n.d. | n.d. | n.d. | 19.8 | 20.5 |
| [MPa] | | | | | | | |
| Viscosity (I) [Pa*s] | 1.2 | 6.5 | 0.2 | 0.2 | 0.3 | 2.5 | 8.2 |
| Viscosity (IV) [Pa*s] | 1.1 | 6.2 | 0.2 | 0.2 | 0.3 | 2.3 | 7.9 |
| tan δ (I) | 1.12 | 0.91 | 1.51 | 1.48 | 1.41 | 0.97 | 1.12 |
| tan δ (IV) | 1.14 | 0.94 | 1.51 | 1.48 | 1.41 | 0.99 | 1.14 |
| Viscosity (I) (6 | 0.9 | 1.8 | n.d. | n.d. | n.d. | 1.1 | 1.8 |
| months, 23° C.) [Pa*s] | | | | | | | |
| Viscosity (IV) (6 | 0.9 | 1.4 | n.d. | n.d. | n.d. | 1.0 | 1.6 |
| months, 23° C.) [Pa*s] | | | | | | | |
| tan δ (I) (6 months, | 1.18 | 1.02 | n.d. | n.d. | n.d. | 1.13 | 1.03 |
| 23° C.) | | | | | | | |
| tan δ (IV) (6 months, | 1.19 | 1.05 | n.d. | n.d. | n.d. | 1.14 | 1.05 |
| 23° C.) | | | | | | | |

TABLE 6C
Comparative Examples 4O-4P

| Comparative Example | 4O | 4P |
|---|---|---|
| Adhesion (dentine) | 28.9 | 25.3 |
| [MPa] | | |
| Adhesion (enamel) | 30.7 | 28.4 |
| [MPa] | | |
| Viscosity (I) [Pa*s] | 60.4 | 58.8 |
| Viscosity (IV) [Pa*s] | 58.9 | 58.4 |
| tan δ (I) | 0.65 | 0.78 |
| tan δ (IV) | 0.88 | 0.83 |
| Viscosity (I) (6 | 22.4 | 15.7 |
| months, 23° C.) [Pa*s] | | |
| Viscosity (IV) (6 | 20.1 | 15.3 |
| months, 23° C.) [Pa*s] | | |
| tan δ (I) (6 months, | 0.54 | 0.69 |
| 23° C.) | | |
| tan δ (IV) (6 months, | 0.68 | 0.75 |
| 23° C.) | | |

1. A dental etching composition comprising
A) an acid selected from the group consisting of phosphoric acid, hydrochloric acid, hydrofluoric acid, maleic acid and citric acid,
B) water, and
C) one or more urethane-urea compounds.

2. The dental etching composition of claim 1, wherein the acid (A) is phosphoric acid or hydrochloric acid.

3. The dental etching composition of claim 1, wherein the urethane-urea compounds (C) have the formula
R¹—O—C(═O)—NH—R²—NH—C(═O)[—NH—R³—NH—C(═O)—NH—R²—NH—C(═O)]ₓ—OR¹
wherein
R¹ is an n-alkyl radical having 4 to 22 carbon atoms, a branched alkyl radical having 4 to 22 carbon atoms, an alkenyl radical having 3 to 18 carbon atoms, a cycloalkyl radical having 3 to 20 carbon atoms, an aryl radical having 6 to 12 carbon atoms, an arylalkyl radical having 7 to 12 carbon atoms, a radical of the formula CₘH₂ₘ₊₁(O—CₙH₂ₙ)ₚ—, CₘH₂ₘ₊₁(OOC—CH₂ᵥ)ₚ—, or R⁵—C₆H₄(O—CₙH₂ₙ)ₚ—, wherein m=1 to 22, n=2 to 4, p=1 to 15, v=4 or 5, and R⁵ is an alkyl radical having 1 to 12 carbon atoms, wherein different R¹ radicals may be the same or different,
R² is a branched or unbranched alkylene radical having 4 to 22 carbon atoms, alkenylene radical having 3 to 18 carbon atoms, alkynylene radical having 2 to 20 carbon atoms, cycloalkylene radical having 3 to 20 carbon atoms, cycloalkenylene radical having 3 to 20 carbon atoms, arylene radical having 6 to 12 carbon atoms, or arylalkylene radical having 7 to 14 carbon atoms, wherein different R² radicals may be the same or different,
R³ is 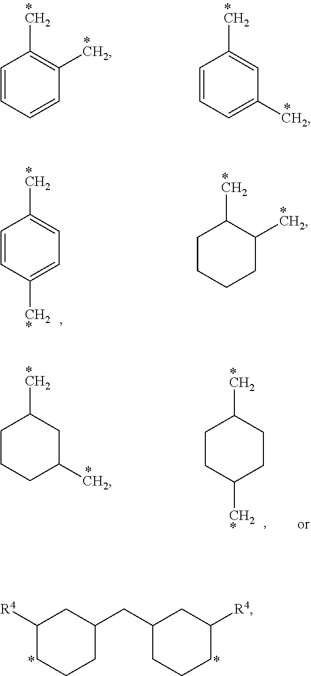
with wherein R⁴=CH₃ or H, and wherein different R³ radicals may be the same or different, and
x is an integer from 1 to 100.

4. The dental etching composition of claim 1, wherein the urethane-urea compounds (C) have the formula
R¹—O—C(═O)—NH—R²—NH—C(═O)[—NH—R³—NH—C(═O)—NH—R²—NH—C(═O)]ₓ—OR¹
wherein
R¹ is an n-alkyl radical having 4 to 10 carbon atoms, a branched alkyl radical having 4 to 10 carbon atoms, an alkenyl radical having 3 to 10 carbon atoms, a cycloalkyl radical having 3 to 10 carbon atoms, an aryl radical having 6 to 12 carbon atoms, an arylalkyl radical having 7 to 12 carbon atoms, a radical of the formula CₘH₂ₘ₊₁(O—CₙH₂ₙ)ₚ—, CₘH₂ₘ₊₁(OOC—CᵥH₂ᵥ)ₚ— or R⁵—C₆H₄(O—CₙH₂ₙ)ₚ—, wherein m=1 to 10, n=2 to 4, p=1 to 15, v=4 or 5, and R⁵ is an alkyl radical having 1 to 12 carbon atoms, a branched alkyl radical having 4 to 10 carbon atoms, a radical of the formula CₘH₂ₘ₊₁(O—CₙH₂ₙ)ₚ-s or CₘH₂ₘ₊₁(OOC—CH₂ᵥ)ₚ—, wherein m=1 to 10, n=2 to 4, p=1 to 15 and v=4 or 5, and wherein different R¹ radicals may be the same or different,
R² is 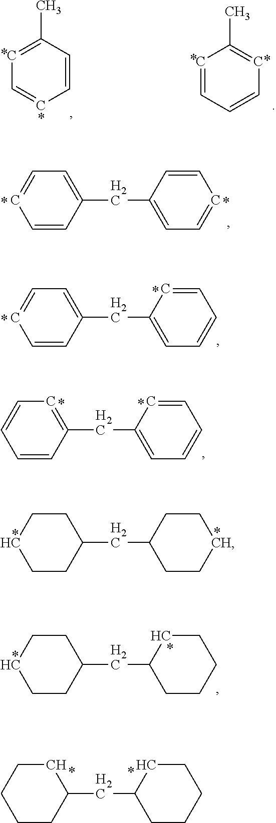
or —(CH₂)_{w}—, wherein w=2 to 10, wherein different R² radicals may the same or different,
R³ is 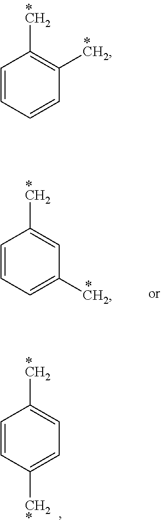
wherein different R³ radicals may be the same or different, and
x is an integer from 1 to 20.

5. The dental etching composition of claim 1, additionally comprising
D) one or more water-miscible solvents.

6. The dental etching composition of claim 5, wherein the water-miscible solvents (D) are selected from the group consisting of ethanol, propan-1-ol, propan-2-ol, butan-1-ol, butan-2-ol, 2-methylpropan-1-ol, 2-methylpropan-2-ol, glycerol, ethylene glycol, propylene glycol, butylene glycol, diethylene glycol, polyethylene glycol, polypropylene glycol, 2-butoxyethan-1-ol, DMSO and acetone, preferably selected from the group consisting of ethanol, propan-1-ol, propan-2-ol, glycerol, polyethylene glycol, polypropylene glycol and DMSO.

7. The dental etching composition of claim 1, additionally comprising
E) colorants.

8. The dental etching composition of claim 7, wherein the colorants (E) are selected from the group consisting of dyes, organic color pigments and inorganic color pigments, and/or wherein the colorants (E) are blue, green or red colorants.

9. The dental etching composition of claim 1, comprising
A) the acid in an amount of 10% to 45% by weight,
B) water in an amount of 30% to 60% by weight,
C) the urethane-urea compounds in an amount of 5% to 20% by weight,
D) a water-miscible solvent in an amount of 0% to 20% by weight, and
E) a colorant in an amount of 0% to 5% by weight,
based in each case on the overall composition.

10. The dental etching composition of claim 1, wherein the composition is essentially free of fumed silica, and/or
wherein the composition does not contain any further constituents apart from (A) the acid, (B) water, (C) the urethane-urea compounds, (D) a water-miscible solvent, and (E) a colorant.

11. The dental etching composition of claim 1, wherein the composition has a loss factor tan δ of less than 1 and/or a viscosity in the range from 0.1 to 200 Pa*s.

12. A method for etching of the hard substance of a tooth, the method comprising etching the hard substance of the tooth with the dental etching composition of claim 1.

13. The method of claim 12, comprising the steps of
i) optionally desiccating the tooth to be treated,
ii) applying the dental etching composition of claim 1 to the hard substance of the tooth to be treated,
iii) allowing a contact time of the dental etching composition to achieve an etching effect on the hard substance of the tooth,
iv) rinsing off the dental etching composition,
v) applying a dental primer composition and/or adhesive composition to the etched hard substance of the tooth,
vi) optionally polymerizing the dental primer composition and/or adhesive composition,
vii) applying a dental restoration composition and
viii) polymerizing the dental restoration composition.

14. The method of claim 13, wherein the hard substance of the tooth is etched in the course of filling treatment.

15. A kit comprising:
the dental etching composition of claim 1,
a dental primer composition and/or adhesive composition, and
optionally a dental restoration composition.

16. The dental etching composition of claim 4, wherein:
R¹ is an n-alkyl radical having 4 to 10 carbon atoms;
R² is 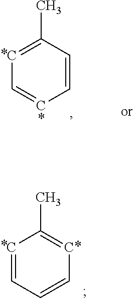
and
x is an integer from 1 to 10.

17. The dental etching composition of claim 9, comprising:
A) the acid in an amount of 30% to 42% by weight,
B) water in an amount of 40% to 60% by weight,
C) the urethane-urea compounds in an amount of 5% to 15% by weight,
D) the water-miscible solvent in an amount of 1% to 15% by weight, and
E) the colorant in an amount of 0.0001% to 1% by weight,
based in each case on the overall composition.

18. The dental etching composition of claim 10, wherein the composition is essentially free of silica particles and inorganic solids.

19. The dental etching composition of claim 11, wherein the composition has a viscosity in the range from 1 to 50 Pa*s.

20. The dental etching composition of claim 11, wherein after storage at 23° C. for 6 months, the composition has a loss factor tan δ of less than 1 and/or a viscosity in the range from 0.1 to 200 Pa*s.
